(19) United States
(12) Patent Application Publication
(10) Pub. No.: US 2019/0375993 A1
(43) Pub. Date: Dec. 12, 2019

(54) OIL SOLUBLE SULFIDE SCAVENGERS WITH LOW SALT CORROSION AND METHODS OF MAKING AND USING THESE SCAVENGERS

(71) Applicant: General Electric Company, Schenectady, NY (US)

(72) Inventors: Hitesh Ghanshyam Bagaria, The Woodlands, TX (US); Gregory Kaplan, Trevose, PA (US)

(21) Appl. No.: 16/462,820

(22) PCT Filed: Dec. 7, 2016
(86) PCT No.: PCT/US2016/065269

§ 371 Date: May 21, 2019

(51) Int. Cl. C10G 29/20; B01D 53/14; B01D 53/52; B01D 53/78; C10L 3/10; C07C 239/12

(52) U.S. Cl. CPC C10G 29/20; B01D 53/1493; B01D 53/1468; B01D 53/52; B01D 53/78; C10G 2300/207; C07C 239/12; B01D 2252/20431; B01D 2252/20405; B01D 2252/20484; C10L 2290/545; C10L 3/103

(57) ABSTRACT

Sulfide scavengers useful to reduce sulfide concentration in fluid streams and methods of using these scavengers. The scavengers comprise oil soluble reaction products of formaldehyde/N-substituted hydroxylamines and can be used to reduce, for example, H₂S content in viscous hydrocarbon oil streams.

### FIELD OF INVENTION

The invention pertains to oil soluble N-substituted hydroxylamine/formaldehyde reaction products (hereinafter sometimes referred to as AHAF), methods of making same and methods of using same to reduce sulfide content in fluid (i.e., gas or liquid) streams.

### BACKGROUND OF THE INVENTION

Hydrogen sulfide or H₂S, is a clear, toxic gas with a foul odor. It is also highly flammable. The Environmental Protection Agency and other regulatory agencies worldwide strictly control the release of H₂S into the environment. H₂S may be present in well water, waste water, and other aqueous systems. H₂S is often present in crude oil and natural gas reserves and must be reduced before making commercial use of such reserves. The H₂S concentration in these reserves prior to treatment typically varies with location and is usually higher in natural gas than in crude oil reserves. In natural gas reserves, for example, H₂S may vary from less than 100 ppm to 3000 ppm. Permitted H₂S levels will also vary by location. The U.S. limits H₂S in natural gas pipelines to 4 ppm per 100 standard cubic feet (0.3 gr/100 scf).

Generally, hydrocarbon streams are treated to reduce sulfides, including organic sulfides, mercaptans, thiols, COS, and H₂S by using chemicals that will react with the sulfides. These chemicals are called scavengers, or sweetening agents.

Monoethanolamine (MEA) triazine is a widely used H₂S scavenger; however, the high amine salt corrosion potential is a major concern for refinery operation. Since it is a water based product, MEA triazine has mass transfer limitations that limit its applications in highly viscous streams.

Most hydrocarbon reserves are treated continuously near the wellhead, though treating hydrocarbons in a batch or similar application elsewhere is not uncommon. Continuous treatment installations near the wellhead inject sulfide scavengers directly into the hydrocarbon pipeline. The injection system typically includes a chemical injection pump and piping tees or atomization nozzles to introduce the scavengers into the pipeline. The amount of scavenger required will vary depending on a variety of factors including the type of scavenger used, the amount of H₂S in the well, permissible H₂S limits, and the well flow rate. Thus, the amount of scavenger added to treat a hydrocarbon pipeline typically ranges from approximately 1 ppm to about 100,000 ppm by volume of the hydrocarbon stream. A length of the pipeline is provided to allow for contact between the scavenger and the sulfide.

### SUMMARY OF THE INVENTION

In certain embodiments, the invention pertains to a method for reducing sulfides in a fluid stream comprising contacting the fluid stream with a N-substituted hydroxylamine/formaldehyde reaction product having the formula

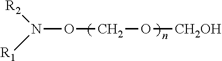

wherein n is an integer from about 0-10 and R₁ and R₂ are each independently chosen from H, C₁-C₁₀ linear, branched, and cyclic alkyl, alkenyl, or aryl groups; with the proviso that both R₁ and R₂ are not H. The sulfides may, for example, comprise one or more members selected from the group consisting of organic sulfides, mercaptans, thiols, COS, and H₂S. The fluid streams may comprise a hydrocarbon stream or an aqueous stream.

From about 1-100,000 ppm by volume of the reaction product is brought into contact with the fluid stream based upon 1,000,000 parts of the fluid stream. In other embodiments, about 500-3,000 ppm of the reaction product is brought into contact with the fluid stream.

In other exemplary embodiments, R₁ and R₂ of the above formula are both C₁-C₁₀ alkyl. In some embodiments, both R₁ and R₂ are ethyl.

Other embodiments of the invention are directed toward methods for making a N-substituted hydroxylamine/formaldehyde reaction product comprising reacting a N-substituted hydroxylamine of the formula RR′NOH with formaldehyde, wherein R and R′ are each independently chosen from H, linear, branched, and cyclic C₁-C₁₀ alkyl, alkenyl, or aryl groups; with the proviso that both R and R′ are not H. The method is conducted at a temperature of about above 60° C. for about 0.5-2.0 hours. In some embodiments, the reaction is conducted in the presence of an organic solvent, and in other embodiments, the reaction is conducted at temperatures of about 80-90° C.

In some embodiments, the N-substituted hydroxylamine is chosen from one or more members selected from the group consisting of N,N-dimethylhydroxylamine, N,N-diethylhydroxylamine, N,N-dibenzylhydroxylamine, N-ethylhydroxylamine, N-propylhydroxylamine, N-isopropylhydroxylamine, N-butylhydroxylamine, N-phenylhydroxylamine, N-cyclohexylhydroxylamine, N-tert-butylhydroxylamine, N-benzylhydroxylamine.

In certain embodiments, the molar ratio of formaldehyde:N-substituted hydroxylamine is from about 0.5-5 moles formaldehyde:N-substituted hydroxylamine. In other embodiments, the molar ratio of formaldehyde:N-substituted hydroxylamine is from about 1-3 moles of formaldehyde:1 mole N-substituted hydroxylamine. In some embodiments, the formaldehyde is in the form of paraformaldehyde.

In other embodiments, the N-substituted hydroxylamine/formaldehyde reaction product (AHAF) has the structure

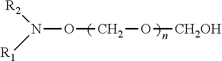

wherein n is an integer from 0-10; R₁ and R₂ are each independently chosen from H, C₁-C₁₀ linear, branched and cyclic, alkyl, alkenyl, or aryl groups; with the proviso that both R₁ and R₂ are not H. In certain embodiments, the reaction product is oil soluble. In some embodiments, R₁ and R₂ are both C₁ -C₁₀ alkyl such as ethyl.

### DETAILED DESCRIPTION

One aspect of the invention pertains to methods of making a N-substituted hydroxylamine/formaldehyde reaction product (AHAF) wherein a N-substituted hydroxylamine of the formula RR′NOH is reacted with formaldehyde (e.g., paraformaldehyde) neat or in the presence of an organic solvent. The reaction may proceed at temperatures of from above about 60° C. for about 0.5-2.0 hours. In certain embodiments, the reaction may be carried out for about 1 hour at temperatures of about 80-90° C. In the above N-substituted hydroxyl amine formula, R and R′ are independently selected from H, linear, branched, and cyclic C₁-C₁₀ alkyl, alkenyl, or aryl groups; with the proviso that both R and R′ are not H. Examples of R and R′ include methyl, ethyl, propyl, butyl, pentyl, hexyl, octyl, and decyl. Particularly noteworthy is diethylhydroxylamine (DEHA).

In some embodiments, the hydroxyamine is chosen from the group consisting of N,N-dimethylhydroxylamine, N,N-diethylhydroxylamine, N,N-dibenzylhydroxylamine, N-ethylhydroxylamine, N-propylhydroxylamine, N-isopropylhydroxylamine, N-butylhydroxylamine, N-phenylhydroxylamine, N-cyclohexylhydroxylamine, N-tert-butylhydroxylamine, N-benzylhydroxylamine.

In those embodiments in which an organic solvent is employed, heavy aromatic naptha solvent may be mentioned as exemplary. The adduct reaction product may remain in the solvent and it can be used as such to reduce sulfide content of hydrocarbon fluid streams, or the adduct can be separated from the reaction medium via conventional separation techniques and then used as a sulfide scavenger. Other organic solvents that may be mentioned include pentane, hexane, cyclohexane, benzene, toluene, chloroform, diethyl ether, dichloromethane, tetrahydrofuran (THF), ethyl acetate, etc. The molar ratio of the reactants, formaldehyde:N-substituted hydroxylamine, may range from about 0.5-5:1 and a ratio of about 1-3:1 can also be mentioned as exemplary.

The AHAF reaction products have the structure

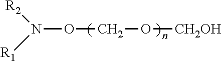

wherein n is an integer from about 0-10; R₁ and R₂ are each independently selected from H, C₁-C₁₀ linear, branched, and cyclic alkyl, alkenyl, or aryl groups; with the proviso that both R₁ and R₂ are not H. In the case wherein DEHA is reacted with formaldehyde (e.g. paraformaldehyde), R₁ and R₂ are both ethyl.

In other aspects of the invention, a method for reducing sulfides from fluid streams is disclosed wherein the AHAF reaction products are brought into contact with such fluid streams that contain one or more organic sulfides, mercaptans, thiols, COS, and H₂S. The fluid streams may include liquid and gas media, and these streams may be hydrocarbon streams or aqueous streams. The reaction products may be employed in amounts of from about 1 to 100, 000 ppm by volume of the fluid stream. Other exemplary dosage ranges that may be mentioned include 500-3,000 ppm, especially about 1,000 ppm.

The AHAF reaction products possess advantage in that they present low risk for amine salt corrosion of metallurgies in contact with the fluid streams and have a higher flash point compared to amines such as dipropylamine and dibutylamine; thus abating safety and handling concerns. The adducts have a low PPI (salt precipitation index) thus reducing salt corrosion risk. The adducts are oil soluble and can therefore be used in heavy, viscous hydrocarbon streams.

In other exemplary embodiments, the fluid stream treated can comprise a fluid hydrocarbon stream or an aqueous fluid stream. These fluid streams may, for example, comprise gas/liquid mixtures from oilfield processes, pipelines, tanks, tankers, refineries, and chemical plants. Additionally, the fluid stream may comprise farm discharge city water, etc. Other additional fluid streams include water, waste water, and process water containing H₂S.

The invention will be further described in connection with the following illustrated examples that should not be construed as limiting the invention.

### EXAMPLES

### Example 1

Formaldehyde: DEHA adduct (2:1 mole ratio), neat. 45 gm of solid paraformaldehyde was placed in the flask. Anhydrous DEHA (65 gm) was added. The mixture was stirred and heated to 90° C. for 1 hour, until paraformaldehyde complete dissolved. Cooled to room temperature, collected 110 gm of adduct (100%).

### Example 2

Formaldehyde: DEHA adduct (2:1 mole ratio), in solvent. 65 gm of solid paraformaldehyde was placed in the flask. Anhydrous DEHA (89 gm) and 51 gm of Aromatic A-150 solvent were added. Mixture was stirred and heated to 90° C. for 1 hour, until paraformaldehyde completely dissolved. Aromatic A-150 is a heavy aromatic solvent naptha. Cooled to room temperature, collected 205 gm of adduct (100%).

### Example 3

Formaldehyde: DEHA adduct (1:1 mole ratio), neat. 23 gm of solid paraformaldehyde was placed in the flask. Anhydrous DEHA (65 gm) was added. Mixture was stirred and heated to 90° C. for 1 hour, until paraformaldehyde completely dissolved. Cooled to room temperature, collected 88 gm of adduct (100%).

### Example 4

Formaldehyde: DEHA adduct (1:1 mole ratio), in solvent. Amount of 33 gm of solid paraformaldehyde was placed in the flask. Anhydrous DEHA 89 gm) and 51 gm of Aromatic A-150 solvent was added. Mixture was stirred and heated to 90° C. for 1 hour, until paraformaldehyde completely dissolved. Cooled to room temperature, collected 173 gm of adduct (100%).

### Example 5

In order to demonstrate the efficacy of the N-substituted hydroxylamine-formaldehyde adducts in reducing H₂S in hydrocarbon media, 150 ml of bunker fuel in 500 ml was mixed with or without sulfide scavenger candidate chemical and heated to 75° C. The headspace H₂S vapor concentration was measured using a stain/dragger tube after 2 hours. The following table shows the resulting data.

TABLE

| | | ppm H₂S @ | Salt Precipitation |
|---|---|---|---|
| Chemical Additive | Dose | 75° C. | Potential Index of Amine |
| Blank | 0 | 900 | NA |
| 40% Glyoxal | 1000 | 500 | NA |
| DMAPA triazine | 1000 | 20 | High |
| Dialkylamine-HCHO adduct | 1000 | 30 | Low |
| HCHO:DEHA adduct (1:1) | 1000 | 0 | Very Low |
| (Example 3 above) | | | |

DMAPA = dimethylaminopropyl amine

While illustrative embodiments of the invention have been described, it should be understood that the present invention is not so limited, and modifications may be made without departing from the present invention. The scope of the invention is defined by the appended claims viewed under either a literal infringement or doctrine of equivalents analysis.

1. A method for reducing sulfides in a fluid comprising contacting said fluid with a reaction product of a N-substituted hydroxylamine and formaldehyde (AHAF).

2. A method as recited in claim 1 wherein said N-substituted hydroxylamine comprises one or more members selected from the group consisting of N,N-dimethylhydroxylamine, N,N-diethylhydroxylamine, N,N-dibenzylhydroxylamine, N-ethylhydroxylamine, N-propylhydroxylamine, N-isopropylhydroxylamine, N-butylhydroxylamine, N-phenylhydroxylamine, N-cyclohexylhydroxylamine, N-tert-butylhydroxylamine, N-benzylhydroxylamine.

3. A method as recited in claim 1 wherein said reaction product (AHAF) has the formula: 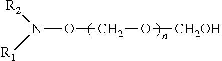
wherein n is from about 0-10 and R₁ and R₂ are each independently chosen from H, C₁-C₁₀ linear, branched, and cyclic alkyl, alkenyl, or aryl groups; with the proviso that both R₁ and R₂ are not H.

4. The method as recited in claim 1 wherein said sulfides comprise one or more members selected from the group consisting of organic sulfides, mercaptans, thiols, COS, and H₂S.

5. The method of claim 1 wherein said fluid is (i) a hydrocarbon, (ii) natural gas, (iii) water, or (iv) a multiphase mixture of hydrocarbon.

6-8. (canceled)

9. The method of claim 5 wherein said hydrocarbon is a hydrocarbon oil selected from the group consisting of crude oil, naptha, gas oil, bunker fuel, marine diesel, asphalt, and bitumen.

10. The method of claim 1 wherein from about 1 to 100,000 ppm by volume of said reaction product is brought into contact with said fluid based upon one million parts of said fluid.

11. (canceled)

12. The method as recited in claim 3 wherein R₁ and R₂ are both C₁-C₁₀ alkyl.

13. The method as recited in claim 12 wherein both R₁ and R₂ are ethyl.

14. A method for making a dialkylhydroxylamine/formaldehyde reaction product comprising reacting a hydroxylamine of the formula RR′NOH with formaldehyde, wherein R and R′ are each independently chosen from H, linear, branched, and cyclic C₁-C₁₀ alkyl, alkenyl, or aryl groups; with the proviso that both R₁ and R₂ are not H; said method being conducted at a temperature of about above 60° C. for about 0.5-2.0 hours.

15. A method as recited in claim 14 wherein said reaction is conducted in the presence of an organic solvent.

16. A method as recited in claim 14 wherein said reaction is conducted at a temperature of about 80-90° C.

17. A method as recited in claim 14 wherein R and R′ are both C₁-C₁₀ alkyl.

18. A method as recited in claim 17 wherein R and R′ are both ethyl.

19. A method as recited in claim 14 wherein the molar ratio of formaldehyde:N-substituted hydroxylamine is from about 0.5-5 moles formaldehyde to about 1 mole of N-substituted hydroxylamine.

20. (canceled)

21. A method as recited in claim 19 wherein said formaldehyde is paraformaldehyde.

22. An N-substituted hydroxylamine/formaldehyde reaction product having the structure 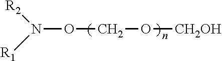
wherein n is an integer from 0-10; R₁ and R₂ are each independently chosen from H, C₁-C₁₀ linear, branched, alkyl, alkenyl, or aryl groups with the proviso that both R₁ and R₂ are not H.

23. The reaction product as recited in claim 22, wherein said reaction product is oil soluble.

24. The reaction product as recited in claim 23 wherein both R₁ and R₂ are C₁-C₁₀ alkyl.

25. The reaction product as recited in claim 24 wherein both R₁ and R₂ are ethyl.
